(19) United States
(12) Patent Application Publication
(10) Pub. No.: US 2025/0109235 A1
(43) Pub. Date: Apr. 3, 2025

(54) POLYMER NANOPARTICLE COMPOSITIONS FOR NON-VIRAL GENE DELIVERY

(71) Applicant: BATTELLE MEMORIAL INSTITUTE, Columbus, OH (US)

(72) Inventors: Cherry GUPTA, Columbus, OH (US); Caleb T. HILLRICH, Columbus, OH (US); Ashlee J. COLBERT, Blacklick, OH (US); Emma K. SCHMITZ, Columbus, OH (US); Joseph GEERS, Columbus, OH (US); Molly KAUFMAN, Columbus, OH (US); Daniel GARBARK, Columbus, OH (US); Danielle J. HUK, Hilliard, OH (US); Matthew Neal, Columbus, OH (US)

(21) Appl. No.: 18/902,038

(22) Filed: Sep. 30, 2024

Related U.S. Application Data
(60) Provisional application No. 63/586,961, filed on Sep. 29, 2023.

(51) Int. Cl. C08F 293/00; B82Y 5/00; C12N 15/88

(52) U.S. Cl. CPC C08F 293/00; C12N 15/88; B82Y 5/00; C08F 2438/03

(57) ABSTRACT

The disclosure relates to block copolymer nanoparticles for in vivo therapeutic delivery, and methods therefor. More particularly, the invention relates to polymer nanoparticles, such as reversible addition-fragmentation chain transfer (RAFT) polymer compositions, for encapsulation and for delivery of large nucleic acids.

### RELATED APPLICATIONS

This application claims the benefit of U.S. Provisional Application No. 63/586,961, filed Sep. 29, 2023, the entire disclosure of which is incorporated herein by reference.

### BACKGROUND

Genetic medicines (including gene therapy, gene silencing, splicing regulators, and nuclease-based gene editors) are poised to produce revolutionary treatments, including vaccines, infectious disease treatments, antimicrobial treatments, antiviral treatments, and most notably, genetic disease treatments. However, the in vivo delivery of these genetic medicine payloads to the specific tissues and cells that need to be treated, while avoiding tissues and cells that can reduce the efficacy or safety of the genetic medicine, poses a significant challenge. Additional challenges include the ability to deliver large genetic payloads or multiple payloads. Adeno-associated viruses (AAVs) are the most widely used tool for genetic medicine delivery, but AAVs are not able to deliver large genetic payloads or multiple payloads (such as the clustered regularly interspaced short palindromic repeats (CRISPR)/Cas9 system), and they sometimes trigger unwanted immune responses, including the generation of anti-AAV antibodies, a cell mediated response. Some of the immune responses caused by AAV in patients are potentially fatal immune responses.

Therapeutics based on the CRISPR/Cas9 system have an exceptional potential to treat a number of genetic diseases due to the capability of this system for precise and programmable gene editing. Gene editing and repair using the CRISPR/Cas9 system has two main mechanisms, including non-homologous end joining (NHEJ) which repairs the site of cut by inducing random indel mutation, and homology-directed repair (HDR), which repairs the cut site based on a pre-existing template. Because a pre-designed template can be used for HDR-directed repair, therapies based on this mechanism can be tailored to cure a large number of different genetic diseases. However, the main challenge is that HDR repair requires the delivery of CRISPR/Cas9, small guide RNA (sgRNA) and a donor DNA strand at the same time to a particular location. This requirement becomes particularly limiting for in vivo applications because ensuring co-delivery of multiple large molecules to the same targeted location is currently not feasible. For example, the Cas9 enzyme sequence and guide RNA complex is too large to fit into AAVs.

Thus, there is a need for effective non-viral delivery systems, including gene delivery systems. The current state-of-the-art non-viral gene delivery systems, such as liposomes, have many drawbacks such as poor biocompatibility and the inability to easily engineer or functionalize them. Additional concerns are that such non-viral gene delivery systems are easily degraded by various enzymes as they pass through intracellular or intercellular compartments, and these systems have not been able to package multiple large payloads.

### SUMMARY

In some aspects, the disclosure provides for a composition comprising a non-viral delivery vehicle comprising one or more nanoparticle forming polymers, and a nucleic acid construct.

In one aspect, a block copolymer comprises a homopolymer first block of monomer units, wherein each monomer unit is represented by formula I:

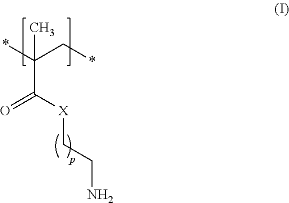

or a salt thereof, wherein:
X is —O— or —NH—; and
p is 0 or an integer selected from 1-3, and

wherein each * represents a point of covalent attachment to the rest of the block copolymer; and

a second block of either:
(i) a homopolymer of monomer units, wherein each monomer unit is represented by formula II:

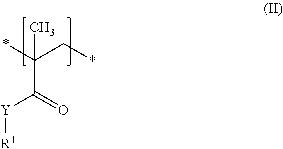

or a salt thereof, wherein:
Y is —O— or —NH—; and
R¹ is C₁₋₆ alkyl or glucose, wherein each hydrogen atom in C₁₋₆ alkyl is optionally substituted by hydroxy, and
wherein each * represents a point of covalent attachment to the rest of the block copolymer; or

(ii) a copolymer comprising two or more monomer units each individually represented by formula II:

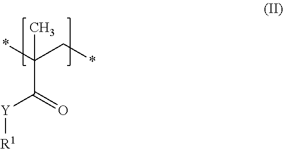

or a salt thereof, wherein:
Y is —O— or —NH—; and
R¹ is C₁₋₆ alkyl or glucose, wherein each hydrogen atom in C₁₋₆ alkyl is optionally substituted by hydroxy, and
wherein each * represents a point of covalent attachment to the rest of the block copolymer; and

wherein the first block is at least about 15 mol % of the block copolymer.

In another aspect, a polymer nanoparticle comprises a block copolymer according to the present disclosure, wherein the nanoparticle has a hydrodynamic diameter of about 30 nm to about 500 nm.

In another aspect, the disclosure provides for a composition comprising a polymer nanoparticle according to the present disclosure and a nucleic acid complexed to the polymer nanoparticle.

In another aspect, a composition according to the present disclosure is useful as a transfection agent for large nucleic acid payloads.

### BRIEF DESCRIPTION OF THE DRAWINGS

FIG. 1 shows an illustration of forming a polymer nanoparticle according to the present disclosure, coupling it to a targeting group, and then complexing the polymer nanoparticle to a nucleic acid.

FIG. 2 shows the encapsulation efficiency of the polymer nanoparticles having first blocks of DMAEMA, DPAEMA, or AEMA, conjugated to different targeting groups (x-axis) for 150 ng of pDNA that is 19.5 kbp.

FIG. 3 shows the transfection efficiency of the polymer nanoparticles having first blocks of DMAEMA, DPAEMA, or AEMA, at a 15:1 PNP:DNA or 25:1 PNP:DNA ratio into human immortalized hTERT Schwann cells.

FIG. 4 shows the transfection efficiency of nanoparticles having first blocks of DMAEMA, DPAEMA, or AEMA into HEK 293T cells using a 4.5 kb EGFP DNA plasmid.

FIGS. 5A-B shows expression of eGFP in the neurons of the cortex in a rat brain from an about 20 kbp eGFP plasmid delivered with a PNP according to the present disclosure.

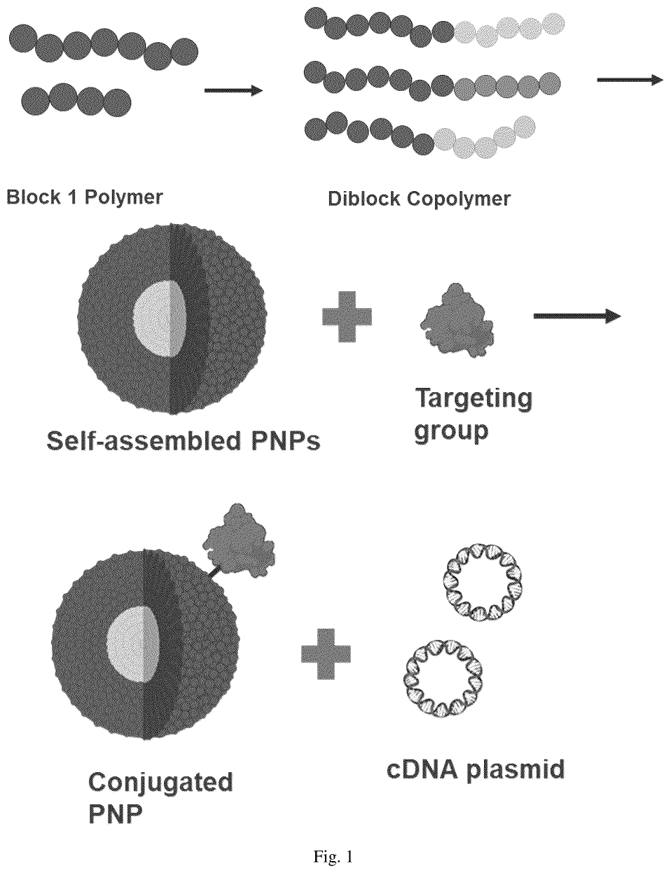

### DETAILED DESCRIPTION

This disclosure describes compositions of cationic polymers (e.g., diblock copolymers) which self-assemble in aqueous conditions to form polymer nanoparticles (PNPs). In illustrative embodiments, the PNPs can encapsulate large nucleic acids (e.g., up to 19.5 kbp or greater), efficiently to form complexes. The complexes can then be used to deliver the nucleic acids into cells (e.g., neuronal cells, such as Schwann cells) in vitro and in the brain cortex region in vivo.

Certain embodiments of this disclosure relate to the use of polymer nanoparticle compositions (e.g., a polymer nanoparticle derived from a controlled living/radical polymerization process, such as RAFT copolymers) as a platform with a high degree of tunability in structure and function, opportunities to protect payloads from adverse reactions or degradation by the immune system, and passive cell targeting via surface charge, or particle size. These delivery systems also lend themselves to computer-aided design, and they have suitable pathways to robust, commercial scale manufacturing processes with higher yields and fewer purification steps than viral delivery composition manufacturing processes.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” may be an approximation of ±10%, ±5%, or ±1%. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**” and “ [symbol] ” each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*”, “A-**”, or

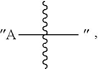

where each of “-*”, “-**”, and

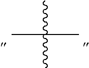

represents a bond to A and the point of covalent bond attachment to B. Alternatively, in some embodiments, the portion of A-B defined by the group or chemical structure B can be represented by, “*-B”, “**-B”, or

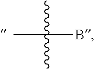

where each of “-*”, “-**”, and

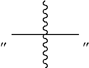

represents a bond to B and the point of covalent bond attachment to A.

As used herein, “molecular weight” refers to the weight average molecular weight (Mw) determined by a conventional polystyrene standard curve gel permeation chromatography method (hereinafter referred to as GPC) using multiple narrow distribution polystyrene standard samples relevant to the present invention and preferably between 2 kDa to 1,000 kDa.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit disease may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a compound or a composition which reduces the frequency of, or delays the onset of, symptoms of a medical condition in a subject relative to a subject which does not receive the compound or composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance (e.g., a compound, a composition, or an agent) to a subject can be carried out using one of a variety of methods known to those skilled in the art. For example, a substance can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A substance (e.g., a compound, a composition, or agent) can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance (e.g., a compound, a composition, or an agent) to a subject will also depend, for example, on the age and/or the physical condition of the subject and the chemical and biological properties of the compound or agent (e.g., solubility, digestibility, bioavailability, stability and toxicity). In some embodiments, a substance (e.g., a compound, a composition, or an agent) is administered orally, e.g., to a subject by ingestion. In some embodiments, the orally administered substance is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents).

For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

### Chemical Definitions

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, OCO—CH₂—O—alkyl, OP(O)(O-alkyl)₂ or CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents. Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “alkenyl” refers to a straight- or branched-chain mono-valent hydrocarbon group having one or more double bonds. In some embodiments, it can be advantageous to limit the number of atoms in an “alkenyl” to a specific range of atoms, such as C₂-C₂₀ alkenyl, C₂-C₁₂ alkenyl, or C₂-C₆ alkenyl. Examples of alkenyl groups include ethenyl (or vinyl), allyl, and but-3-en-1-yl. Included within this term are cis and trans isomers and mixtures thereof. It will be appreciated that an alkenyl can be unsubstituted or substituted as described herein. An alkenyl group can be substituted with any of the substituents in the various embodiments described herein, including one or more of such substituents.

The term “C_{x,y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀ alkyl indicates a hydrogen where the group is in a terminal position, a bond if internal. A C₁₀₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

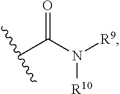

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

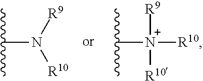

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “carboxy”, as used herein, refers to a group represented by the formula CO₂H.

The term “ester”, as used herein, refers to a group C(O)OR⁸ wherein R⁸ represents a hydrocarbyl group.

The term “ketone”, as used herein, refers to a group C(O)R⁷ wherein R⁷ represents a hydrocarbyl group (e.g., alkyl, aryl, heteroaryl).

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The term “hydrocarbyl”, as used herein, refers to a group that is bonded through a carbon atom that does not have a═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, aphosphoryl, aphosphate, aphosphonate, aphosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The nomenclature “(ATOM)ᵢ.(ATOM)ⱼ” with j>i, when applied herein to a class of substituents, is meant to refer to embodiments of this disclosure for which each and every one of the number of atom members, from i to j including i and j, is independently realized. By way of example, the term C₁-C₃ refers independently to embodiments that have one carbon member (C₁), embodiments that have two carbon members (C₂), and embodiments that have three carbon members (C₃).The term “modulate” as used herein includes the inhibition or suppression of a function or activity (such as cell proliferation) as well as the enhancement of a function or activity.

“Acceptable salt” or “salt” is used herein to refer to an acid addition salt or a basic addition salt which is suitable for or compatible with the compounds or a desired treatment.

The disclosure also includes acceptable salts (e.g., pharmaceutically acceptable salts) of the block copolymers described herein, preferably of those described above and of the specific compounds exemplified herein, and pharmaceutical compositions comprising such salts, and methods of using such salts.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

It will be understood that the chemical entities described herein, can exist as a salt of a free acid or base of a compound represented herein and an inorganic or organic counter ion. Illustratively, the salt can be formed during the manufacture of the compound described herein (e.g., a salt or a pharmaceutically acceptable salt) or a compound described herein can be substituted to provide a salt for further manufacture, formulation, or administration reasons. As illustrated herein, certain compounds include a “W^{⊖},” wherein “W^{⊖}” is an inorganic counter ion (e.g., an inorganic anion) or an organic counter ion (e.g., an organic anion). In certain embodiments, W^{⊖} (is an anion that is complexed with a cation of a compound of the disclosure to form a pharmaceutically acceptable salt. As illustrated herein, certain compounds include a “Z⁺,” wherein “Z⁺” is an inorganic counter ion (e.g., an inorganic cation) or an organic counter ion (e.g., an organic cat). In certain embodiments, Z⁺ is a cation that is complexed with an ion of a compound of the disclosure to form a pharmaceutically acceptable salt.

The term “acceptable acid addition salt” as used herein means any non-toxic organic or inorganic salt of any base compounds of the present disclosure. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of the present disclosure are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of the present disclosure for laboratory use, or for subsequent conversion to a pharmaceutically acceptable acid addition salt.

The term “acceptable basic addition salt” as used herein means any non-toxic organic or inorganic base addition salt of any acid compounds represented by compounds or polymers of the present disclosure or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The term “inorganic counter ion” represents an inorganic ion that accompanies an ionic species in order to maintain electric neutrality. An inorganic counter ion may represent an anion or cation. An inorganic counterion may accompany a free acid or base of a compound represented herein. An inorganic ion may form by a reaction of an inorganic base or inorganic acid and a compound described herein that possesses a sufficiently acidic group, a sufficiently basic group, both types of functional groups, or more than one of each type.

The term “organic counter ion” represents an organic counter ion that accompanies an ionic species in order to maintain electric neutrality. The organic ion may represent an anion or cation. An organic counter ion may accompany a free acid or base of a compound represented herein. An organic ion may form by a reaction of an organic base or organic acid and a compound described herein that possesses a sufficiently acidic group, a sufficiently basic group, both types of functional groups, or more than one of each type.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a block copolymer that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

As used herein and in connection with chemical structures depicting the various embodiments described herein, a polymer may be arranged in any linear or branched configuration.

For example, a polymer comprising one or more monomer units may be arranged as a homopolymer, a block copolymer, a statistical copolymer, a random copolymer, or an alternate copolymer of different monomers linked together in an alternating fashion. In one embodiment, the polymers described herein may include repeating blocks, such as in a block copolymer, of units selected from formula I and formula II.

The term “block copolymer” is known in the art, and a representative definition is that a block copolymer is a polymer comprising molecules in which there is a linear arrangement of blocks, for example a block A linearly connected to a block B, where each of the blocks (e.g., block A and block B) comprises units derived from a characteristic species or combination of species of monomer such that at least one difference exists between the species of monomer(s) of the different blocks (e.g., the monomer species or combination of species of block A is different than the monomer species or combination of species of block B.

The term “statistical copolymer” is known in the art, and a representative definition is that a statistical copolymer can be a copolymer composed of monomers that form a sequence based on a statistical rule (e.g., Markovian statistics).

The term “random copolymer” is known in the art, and a representative definition is that a random copolymer describes a copolymer where the probability of finding a given type monomer residue at a particular point in the chain is equal to the mole fraction of that monomer residue in the chain and is independent of the neighboring units in the chain.

The term “alternate copolymer” is known in the art, and a representative definition is that an alternate copolymer describes a copolymer of monomers sequentially linked in a uniform pattern.

“Nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “polynucleotide,” as used herein, means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose. As used herein, a polyribonucleotide sequence that recites thymine (T) is understood to represent uracil (U).

“Polydeoxyribonucleotides,” “deoxyribonucleic acids,” and “DNA” mean macromolecules that include multiple deoxyribonucleotides that are polymerized via phosphodiester bonds. “Polyribonucleotides,” “ribonucleic acids,” and “RNA” mean macromolecules that include multiple ribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP) dNTPs, and may include detectable tags, such as protein tags, luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof).

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini.

The term “amino acid sequence” refers to a series of two or more amino acids linked together via peptide bonds wherein the order of the amino acids linkages is designated by a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA. DNA and RNA can be synthesized naturally (e.g. by DNA replication or transcription of DNA or RNA, respectively). DNA and RNA can also be chemically synthesized. RNA can be post-transcriptionally modified. The terms “target mRNA” and “target transcript,” “target sequence” are synonymous as used herein.

As used herein, “target sequence” refers to a contiguous portion of the nucleotide sequence of an mRNA molecule formed during the transcription of a gene, including mRNA that is a product of RNA processing of a primary transcription product.

“Targeting” an oligomeric compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

The term “RNAi” and “RNA interfering” with respect to an agent of the invention, are used interchangeably herein. RNAi molecules are typically comprised of a sequence of nucleic acids or nucleic acid analogs, specific for a target gene. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA).

As used herein, “shRNA” or “small hairpin RNA” (also called stem loop) is a type of RNAi. shRNAs may be composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs functions as RNAi but are further defined in that shRNA species are double stranded hairpin-like structure for increased stability. These shRNAs, as well as other such agents described herein, can be contained in plasmids, retroviruses, and lentiviruses and expressed from, for example, the pol III U6 promoter, or another promoter.

### Representative Embodiments

This disclosure describes compositions of cationic polymers (e.g., diblock copolymers) which self-assemble in aqueous conditions to form polymer nanoparticles (PNPs). In illustrative embodiments, the PNPs can complex nucleic acids and deliver the complexed nucleic acids to a desired location.

In certain embodiments, a block copolymer comprises a first block and a second block. The first block may comprise a homopolymer of poly aminoethyl methacrylamide (AEMA). The second block may comprise copolymer, such as a statistical copolymer or a random copolymer. For example, the second block may comprise a homopolymer of butylmethacrylate, hydroxyethylmethacrylate, or 2-deoxy-2-methacrylamido glucopyranose (MAG). In certain preferred embodiments, the block copolymer is a diblock copolymer.

In one aspect, a block copolymer comprises a first block and a second block that is different from the first block. The first block can be a homopolymer of monomer units, wherein each monomer unit is represented by formula I:

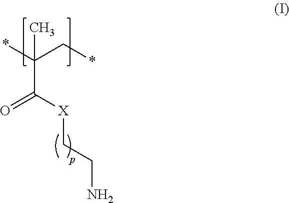

or a salt thereof, wherein:
X is —O— or —NH—; and
p is 0 or an integer selected from 1-3, and
wherein each * individually represents a point of covalent attachment to the rest of the block copolymer; and a second block of either:

(i) a homopolymer of monomer units, wherein each monomer unit is represented by formula II:

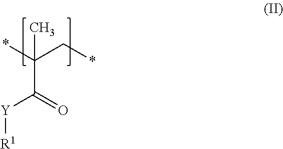

or a salt thereof, wherein:
Y is —O— or —NH—; and
R¹ is C₁₋₆ alkyl or glucose, wherein each hydrogen atom in C₁₋₆ alkyl is optionally substituted by hydroxy, and
wherein each * individually represents a point of covalent attachment to the rest of the block copolymer; or

(ii) a copolymer comprising two or more monomer units each individually represented by formula II:

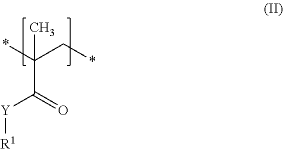

or a salt thereof, wherein:
Y is —O— or —NH—; and
R¹ is C₁₋₆ alkyl or glucose, wherein each hydrogen atom in C₁₋₆ alkyl is optionally substituted by hydroxy, and
wherein each * individually represents a point of covalent attachment to the rest of the block copolymer; and

wherein the first block is at least about 15 mol % of the block copolymer.

In one aspect, a block copolymer comprises a first block and a second block that is different from the first block. The first block can be a homopolymer of monomer units, wherein each monomer unit is represented by formula I:

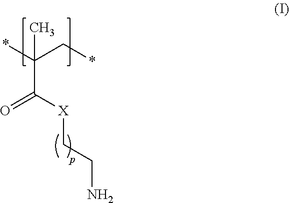

or a salt thereof, wherein X is —O— or —NH—, p is 0 or an integer selected from 1-3 and each * individually represents a point of covalent attachment to the rest of the block copolymer.

In some embodiments, the second block is a homopolymer of monomer units, wherein each monomer unit is represented by formula II:

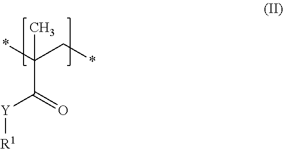

or a salt thereof, wherein:
Y is —O— or —NH—; and
R¹ is C₁-C₆ alkyl or glucose, wherein each hydrogen atom in C₁-C₆ alkyl is optionally substituted by hydroxy, and wherein each * individually represents a point of covalent attachment to the rest of the block copolymer.

In some embodiments, the second block is a copolymer comprising two or more monomer units each individually represented by formula II:

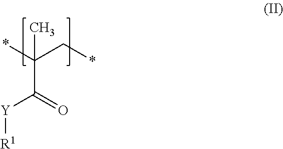

or a salt thereof, wherein:
Y is —O— or —NH—; and
R¹ is C₁-C₆ alkyl or glucose, wherein each hydrogen atom in C₁-C₆ alkyl is optionally substituted by hydroxy, and
wherein each * represents a point of covalent attachment to the rest of the block copolymer.

In some embodiments, p is 0. In some embodiments, p is an integer selected from 1-3, and is preferably 1.

In some embodiments, X is —O—. In some preferred embodiments, X is —NH—.

In some embodiments, the monomer unit of the first block is preferably

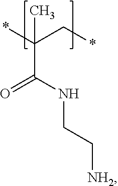

or a salt thereof, wherein each * represents a point of covalent attachment to the rest of the block copolymer. Illustratively, the monomer unit of the first block is derivable from the polymerization of 2-aminoethylmethacrylamide

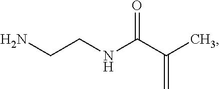

preferably the HCl salt thereof).

In some embodiments, Y is —O— or —NH—. In some preferred embodiments, Y is —O—. In some alternative embodiments, Y is —NH—.

In some embodiments, R¹ is C₁-C₆ alkyl or glucose. Illustratively each hydrogen atom in C₁-C₆ alkyl is optionally substituted by hydroxy. In some embodiments, R¹ is C₁-C₆ alkyl (e.g., C₂-C₆ alkyl or C₃-C₆ alkyl) and is optionally substituted with one hydroxy or is unsubstituted. For example, in some preferred embodiments, R¹ is C₂-C₆ alkyl substituted with hydroxy, for example 2-ethanol (i.e., *—CH₂—CH₂—OH, where the * is the point of covalent attachment to Y). In some embodiments, R¹ is C₃-C₆ alkyl, such as a straight chain C₃-C₆ alkyl, which may be optionally substituted as described herein. In certain preferred embodiments, R¹ is C₃-C₆ alkyl (e.g., C₄ alkyl such as n-butyl) and is unsubstituted.

In some embodiments, R¹ is a glucose

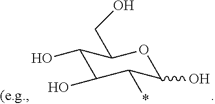 [SMILES: *C1C(O)OC(CO)C(O)C1O]

In certain preferred embodiments when R¹ is a glucose

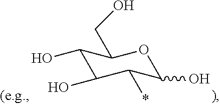 [SMILES: *C1C(O)OC(CO)C(O)C1O]

Y is —NH—. In certain embodiments, where R¹ is a glucose, the monomer unit is derivable from the polymerization of 2-deoxy-2-methacrylamido-D-glucose

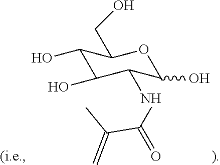

In some embodiments, the second block comprises a homopolymer of MAG.

In another aspect, a block copolymer comprises a first block and a second block, wherein: the first block comprise a homopolymer poly aminoethyl methacrylate (AEMA), and the second block comprises either:
(a) a homopolymer of polybutylmethacrylate (BMA), hydroxyethylmethacrylate (HEMA), or 2-deoxy-2-methacrylamido glucopyranose (MAG) or
(b) a copolymer of at least two of BMA, HEMA, and MAG, wherein the first block has a molecular weight (Mw) of at least 9,000 Da.

In certain embodiments, the second block comprises a homopolymer. For example, in some embodiments, the second block comprises a homopolymer of BMA. In some other embodiments, the second block comprises a homopolymer of HEMA.

In certain embodiments, the second block comprises a copolymer, for example a random copolymer. For example, certain embodiments comprises a copolymer of BMA and HEMA.

In some embodiments, the first block is at least about 15 mol % of the block copolymer. In some embodiments, the first block is at least about 20 mol %, at least about 45 mol %, or at least about 70 mol % of the block copolymer. In some embodiments, the first block is about 20 mol % to about 30 mol %, about 45 mol % to about 55 mol %, or about 70 mol % to about 80 mol % of the block copolymer.

In some embodiments, the second block is less than about 80 mol % of the block copolymer. In some embodiments, the second block is less than about 55 mol % or less than about 30 mol % of the block copolymer. In some embodiments, the second block is about 70 mol % to about 80 mol %, about 45 mol % to about 55 mol %, or about 20 mol % to about 30 mol % of the block copolymer.

In some embodiments, the first block has a mass-averaged molecular weights ranging from about 5 kDa to about 20 kDa or about 5 kDa to about 15 kDa. In some embodiments, the second block has mass-averaged molecular weights ranging from about 5 kDa to about 20 kDa or about 5 kDa to about 15 kDa.

In some embodiments, a block polymer as described herein has one or more of an overall molecular weight (Mₙ) (i.e., the total of all blocks) in the range of about 1 kDa to about 1000 kDa, or about 2 kDa to about 500 kDa, or about 2 kDa to about 160 kDa, and overall degree of polymerization in the range of about 10 to about 3500, or about 20 to about 2500, or about 30 to about 900, a size in the range of about of about 10 nm to about 10000 nm, and a maximum corona-to-core ratio (CCR) of about 1 to about 4. In some embodiments, the overall molecular weight (Mₙ) in the range of about 30 kDa to about 120 kDa, about 40 kDa to about 110 kDa about 50 kDa to about 100 kDa, about 60 kDa to about 90 kDa, about 40 kDa to about 80 kDa, and about 40 kDa to about 60 kDa. In some embodiments, the overall degree of polymerization in the range of about 40 to about 850, about 60 to about 800, about 100 to about 700, about 200 to about 600, or about 300 to about 500. In some embodiments, the size is in the range of about of about 10 nm to about 10000 nm, or about 20 nm to about 5000 nm, or about 50 nm to about 3000 nm, or about 20 nm to about 1000 nm, or about 50 nm to about 1000 nm, or about 30 nm to about 500 nm, or about 200 nm to about 2000 nm, or about 100 nm to about 5000 nm, or about 100 nm to about 500 nm, or about 10 nm to about 50 nm, about 15 nm to about 45 nm, about 20 nm to about 40 nm, or about 25 nm to about 35 nm. In some embodiments, the maximum corona-to-core ratio (CCR) is less than 4, or less than 3, about 1 to about 3.8, about 1.2 to about 3.5, about 1.5 to about 3, about 1.5 to about 2.5, or about 1 to about 2.

In some embodiments, a first block can be prepared from one or more monomer units and have a molecular weight (Mₙ) in the range of about 1 kDa to about 500 kDa, or about 10 kDa to about 200 kDa, or about 10 kDa to about 160 kDa, or about 1 kDa to about 80 kDa, and a degree of polymerization in the range of about 10 to about 3500, or about 10 to about 2500, or about 20 to about 2000, or about 50 to about 1200, or about 50 to about 1000. In some embodiments, a first block molecular weight (Mₙ) can be in the range of about 1 kDa to about 500 kDa, or about 2 kDa to about 400 kDa, or about 5 kDa to about 200 kDa, or about 10 kDa to about 160 kDa, or about 15 kDa to about 100 kDa, or about 25 kDa to about 60 kDa, or about 30 kDa to about 55 kDa, about 30 kDa to about 50 kDa, or about 30 kDa to about 40 kDa, and the like. In some embodiments, the first block degree of polymerization is in the range of about 30 to about 350, about 50 to about 300, about 70 to about 250, about 80 to about 240, about 100 to about 200, and the like.

In some embodiments, the second block can be prepared from one or more monomer units, and can have a molecular weight (Mₙ) in the range of about 1 kDa to about 500 kDa, or about 10 kDa to about 200 kDa, or about 10 kDa to about 160 kDa, or about 1 kDa to about 80 kDa, and a degree of polymerization in the range of about 10 to about 3500, or about 10 to about 2500, or about 20 to about 2000, or about 50 to about 1200, or about 50 to about 1000. In some embodiments, the second block molecular weight (Mₙ) is in the range of about 10 kDa to about 70 kDa, about 15 kDa to about 65 kDa, about 20 kDa to about 60 kDa, about 25 kDa to about 55 kDa, about 30 kDa to about 50 kDa, about 35 kDa to about 45 kDa, about 5 kDa to about 15 kDa, and the like. In some embodiments, the second block degree of polymerization is in the range of about 3 to about 2500; or about 20 to about 2000, or about 30 to about 1500, or about 40 to about 1200, or about 10 to about 500, or about 12 to about 450, or about 20 to about 400, or about 25 to about 350, or about 50 to about 300, or about 100 to about 250, or about 150 to about 200, or about 5 to about 50, or about 5 to about 20, and the like.

In some embodiments, block copolymers as prepared herein can be described by the following structure:

CTACap-[first block 1]ₘ-[second block]ₙ-CTACap

where each CTACap is a capping unit derived from the chain transfer agent(s) used in the process for preparing the RAFT copolymer. The CTA used for preparing each of the first block and the second block can be the same or different. In some embodiments, the CTA used to prepare each the first block and the second block is the same (e.g., macroCTA). In some embodiments, the CTA used to prepare each of the first block and the second block is different. In some embodiments, the CTA used to prepare one or both of the first block and the second block comprises a functional group for the covalent attachment of a biomolecule, drug, or label to the block copolymer. In some embodiments, the covalent attachment can be via an ester or an amide bond. In some embodiments, the covalent attachment can be via EDC-NHS chemistry.

In some embodiments, the first block comprises a cap of formula:

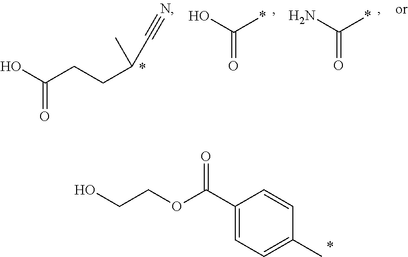

or a salt thereof, wherein * represents a point of covalent attachment to the first block. In certain preferred embodiments, the cap is

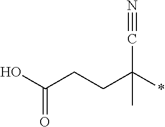 [SMILES: *C(C)(C#N)CCC(=O)O]

In some embodiments, the second capping unit is of formula

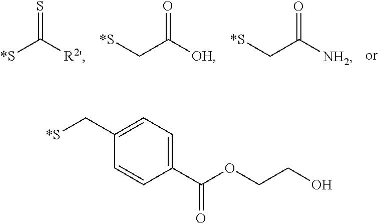

or a salt thereof, wherein * represents a point of covalent attachment to the second block, and R^{2′} is —SC₂-C₁₂ alkyl or C₆H₅, and is preferably

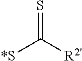

such as

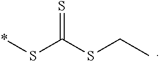 [SMILES: *SC(=S)SCC]

In certain preferred embodiments, the composition comprises 2-aminoethylmethacrylamide (AEMA) as the first block. The second block of these compositions is composed of a subset or combination of butyl methacrylate (BMA), 2-hydroxyethyl methacrylate (HEMA), 2-deoxy-2-methacrylamido glucopyranose (MAG) in the targeted synthesis ratios of 1:1, 1:3 and 3:1 of the first block and the second block.

In certain preferred embodiments, the block copolymer has an AEMA homopolymer first block having a molecular of about 5 kDa to about 30 kDa. The second block is either (ia) a BMA homopolymer having a molecular weight of about 7 kDa to about 60 kDa, (ib) a HEMA homopolymer having a molecular weight of about 8 kDa to about 60 kDa, or (ii) a BMA (about 10 to about 90%) and HEMA (about 90 to about 10%) copolymer, having a molecular weight of about 8 kDa to about 60 kDa. The Zeta potential (mV) is about 20 to about 60. When encapsulating a large nucleic acid (e.g., greater than 15 kbp, such as about 20 kbp), the encapsulation efficiency is greater than about 99%. The transfection efficiency using a large nucleic acid (e.g., greater than 15 kbp, such as about 20 kbp) in neuronal cells is at least about 2%, for example about 2% to about 15% or about 3% to about 15%.

In some embodiments, a polymer nanoparticle comprises a block copolymer according to the present disclosure. In some embodiments, the block copolymer self-assembles into the nanoparticle.

In some embodiments, the nanoparticle has a hydrodynamic diameter (e.g., Z-averaged hydrodynamic diameter) of about 20 nm to about 600 nm, about 30 nm to about 500 nm, or about 50 nm to about 500 nm, or about 100 nm to about 300 nm, as measured by DLS.

In certain embodiments, the nanoparticle is monodispersed, as measured using dynamic light scattering (DLS). In some embodiments, the nanoparticle has a size PDI of about 0.15 to about 0.3, as measured by DLS. In some embodiments, the nanoparticle is multimodal, for example the nanoparticle may be didispersed (e.g., two distinct peaks), as measured by DLS. In certain embodiments, the nanoparticle has a polydispersity index (PDI) of lower than about 0.3. For example, the nanoparticle can have a PDI of about 0.05 to about 3, about 0.1 to about 0.3, about 0.1 to about 0.2, or about 0.2 to about 0.3.

In certain embodiments, the block copolymer has a Zeta potential (mV) of about 15 to about 65. For example, the Zeta potential (mV) can be about 20 to about 60, about 30 to about 60, or about 35 to about 60.

In some embodiments, the nanoparticle comprises a barcode construct covalently attached to the nanoparticle, as described in U.S. Patent Application Publication No. 2022/033309, the entirety of which is hereby incorporated by reference. As described in U.S. Patent Application Publication No. 2022/033309, the presence of the barcode construct can allow for identification of PNPs of interest, for example by determining the presence of a PNP in particular tissue or by performance in an assay. It should be understood that although certain examples in this application are performed on PNPs that include the barcode, similar performance would be expected by PNPs that lack the barcode. polymer nanoparticle.

In one illustrative aspect, the polymer nanoparticles may be used as delivery vehicles according to the present disclosure. In some embodiments, the non-viral delivery vehicle comprises one or more nanoparticle forming polymers. In some embodiments, the non-viral delivery vehicle comprises polymer nanoparticles. In some embodiments, the non-viral delivery vehicle is not a lipid based system. In some embodiments, the non-viral delivery vehicle comprises polymer nanoparticles made from controlled living/radical polymerization processes. It will be appreciated that the identity of the monomer units is not particularly limited so long as the monomer units being used are compatible with a controlled living/radical polymerization, such as reversible-deactivation radical polymerization, atom transfer radical polymerization (ATRP), reversible addition fragmentation chain transfer polymerization (RAFT), iodine-transfer polymerization (ITP), selenium-centered radical-mediated polymerization, telluride-mediated polymerization (TERP), stibine-mediated polymerization, ring-opening polymerization, or like polymerization processes. In some embodiments, the polymer nanoparticles may be made by RAFT copolymerization to synthesize a diverse set of block copolymers, and to screen their ability to form complexes with a payload. In one aspect, polymer nanoparticles (e.g., RAFT copolymers) may be produced by chemically bonding a payload to a constituent polymer, such as by the grafting of the payload onto RAFT copolymers using chain transfer agents, and subsequently assembling the polymers into a delivery vehicle.

In some embodiments, a targeting group is coupled to the polymer nanoparticle, for example through a covalent bond or through electrostatic interaction. The targeting group may be present so that the polymer nanoparticle is directed to a preferred cell, tissue, or location. In some embodiments, the targeting group is selected from HIV TAT cell penetrating peptide, botulinum toxin A, and cholera toxin subunit B.

In some embodiments, the polymer nanoparticle comprises a sulfonated cholesterol (e.g., a PEG-terminated cholesterol, such a sulfonated PEG terminated cholesterol). The sulfonated cholesterol may be electrostatically bound to the polymer nanoparticle. In some embodiments, the hydroxyl group of the cholesterol has been modified to hold a negatively charged sulfonic acid group in the sulfonated PEG-terminated cholesterol. In some embodiments, the cholesterol (e.g., the sulfonated PEG-terminated cholesterol) is of the formula:

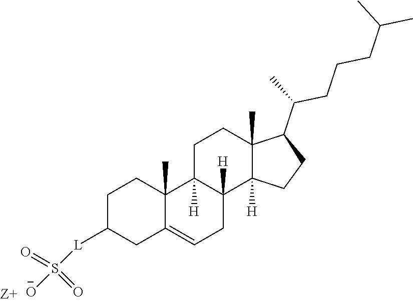

wherein: L is —O— or —OC(O)(C₁-C₆ alkyl)C(O)—NH—(PEG)CH₂CH₂O—, wherein the PEG has a molecular weight of about 800 to about 10K Da, and Z⁺ is a cation (e.g., an ammonium). In certain embodiments, the mass ration of cholesterol:PNP is about 1:20 to about 1:40, preferably about 1:30.

In some embodiments, a composition comprises a polymer nanoparticle as described herein (e.g., may contain a targeting group, a barcode construct, etc.) and a nucleic acid (sometimes called a payload) complexed to the polymer nanoparticle. For example, the nucleic acid may be complexed to the nanoparticle through electrostatic interactions. In certain preferred embodiments, the polymer nanoparticle can serve as a transfection agent to deliver a nucleic acid (for example a large nucleic acid being at least 5 kbp, at least 10 kbp, or at least 15 kbp) to a cell. For example, the transfection efficiency is at least about 0.1%, at least about 0.3%, at least about 0.5%, at least about 0.7%, or at least about 0.9%. As another example, the transfection efficiency is at least about 0.1%, at least about 0.3%, at least about 0.5%, at least about 0.7%, or at least about 0.9% into neuronal cells such as Schwann cells. As another example, the transfection efficiency of a large nucleic acid (i.e., at least 10 kbp or at least 15 kbp) is at least about 0.1%, at least about 0.3%, at least about 0.5%, at least about 0.7%, or at least about 0.9% into neuronal cells such as Schwann cells. In some embodiments, the transfection efficiency (e.g., at a PNP:DNA ratio of about 7.5:1, about 10:1, about 15:1, about 25:1, about 40:1, or about 50:1 by mass) is at least about 1%, at least about 3%, or about least about 9% in cells (e.g., neuronal cells such as Schwann cells). For example, the transfection efficiency (e.g., at a PNP:DNA ratio of about 10:1, about 15:1, or about 30:1 by weight) may be about 1% to about 20%, about 1% to about 18%, about 1% to about 12%, about 1% to about 10%, about 1% to about 6%, about 1% to about 5%, about 2% to about 18%, about 2% to about 16%, about 5% to about 16%, or about 10% to about 20% in cells (e.g., neuronal cells such as Schwann cells). For example, the transfection efficiency may be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% in cells (e.g, neuronal cells such as Schwann cells).

In certain embodiments, the nucleic acid is an RNA (e.g., an mRNA, a microRNA, antisense oligonucleotides, or an siRNA) or a DNA (e.g., an ssDNA, a dsDNA, a complimentary coding DNA (cDNA), or a DNA plasmid (pDNA)). In certain embodiments, the nucleic acid is an mRNA. In certain embodiments, the nucleic acid is a circular RNA. In certain embodiments, the nucleic acid is a DNA plasmid (pDNA).

In some embodiments, the nucleic acid is at least 5 kbp, at least 10 kbp, or at least 15 kbp. In some embodiments, the nucleic acid is about 5 kbp to about 25 kbp, about 10 kbp to about 25 kbp, or about 15 kbp to about 25 kbp.

The polymer nanoparticles described herein are capable of interacting with (e.g., encapsulating) nucleotide plasmids. In some embodiments, the encapsulation efficiency is greater than about 70%, greater than about 75%, greater than about 80%, greater than about 85%, greater than about 90%, greater than about 95%, or greater than about 97%. In certain embodiments, the encapsulation efficiency of a large plasmid greater than 10 kbp is at least about 70%, at least about 75%, at least about 80%, at least about 85%, at least about 90%, at least about 95%, or at least about 97%. In certain embodiments, the encapsulation efficiency of a large plasmid greater than 15 kbp is at least about 70%, at least about 75%, at least about 80%, at least about 85%, at least about 90%, at least about 95%, or at least about 97%. In certain embodiments, the encapsulation efficiency of a large plasmid greater than 19 kbp is at least about 70%, at least about 75%, at least about 80%, at least about 85%, at least about 90%, at least about 95%, or at least about 97%.

In some embodiments, the polymer nanoparticles have low toxicity to cells. For example, in some embodiments, the cell viability after transfection of cells (e.g., neuronal cells such as Schwann cells) is at least about 70%, at least about 75%, at least about 80%, at least about 85%, at least about 90%, or at least about 95%.

The compositions described herein can be used for treating disease. For example, the polymer nanoparticles may be able to deliver a nucleic acid (i.e., a payload) that provides a therapeutic benefit to a patient. In certain embodiments, the polymer nanoparticle is able to deliver the nucleic acid to cells, such as Schwann cells or embryonic cells. The nucleic acids delivered by the polymer nanoparticle may be useful in treating diseases such as Neurofibromatosis type 1 (NF-1), but may be applied for the delivery of therapeutically cargo for other neurological diseases including but not limited to multiple sclerosis, Charcot-Marie-Tooth disease, Parkinson's, Alzheimer's, Amyotrophic Lateral Sclerosis and or other situations where delivery of large genetic payload is warranted, for example to embryonic cells. In certain embodiments, the polymer nanoparticles are capable of delivering nucleic acids (e.g., large nucleic acids) in vivo. The nucleic acids delivered by the polymer nanoparticle may be useful in treating diseases such as Neurofibromatosis type 1 (NF-1) or type 2 (NF-2), but may be applied for the delivery of therapeutically cargo for other neurological diseases including but not limited to multiple sclerosis, Charcot-Marie-Tooth disease, Parkinson's, Alzheimer's, Amyotrophic Lateral Sclerosis, Duchenne Muscular Dystrophy, and or other situations where delivery of large genetic payload is warranted, for example to embryonic cells. In certain embodiments, the polymer nanoparticles are capable of delivering nucleic acids (e.g., large nucleic acids) in vivo, for example to the brain.

In one embodiment a composition comprising a polymer nanoparticle (e.g., a polymer nanoparticle derived from a controlled living/radical polymerization process, such as RAFT polymer) associated with a nucleic acid construct is provided. In another embodiment, a method of in vivo screening to identity a desired polymer nanoparticle (e.g., a polymer nanoparticle derived from a controlled living/radical polymerization process, such as RAFT polymer) associated with a nucleic acid construct for use as a delivery vehicle is provided. In another embodiment, a method of treating a patient with a disease is provided comprising administering to the patient the polymer nanoparticle identified in the screening method.

In some embodiments, the method of in vivo screening for a desired polymer nanoparticle for use as a delivery vehicle comprises, (a) preparing a library comprising two or more types of polymer nanoparticles, wherein each polymer nanoparticle is associated with a nucleic acid construct comprising a different polynucleotide barcode, (b) administering the library to an animal, (c) removing cells or tissues from the animal, (d) isolating the nucleic acid constructs from the cells or tissues of the animal, (e) detecting the nucleic acid constructs in the cells or tissue of the animal, and (f) identifying the desired polymer nanoparticle for use as a delivery vehicle. In various embodiments, the nucleic acid construct can be detected by, for example, the polymerase chain reaction (PCR), isothermal amplification, or sequencing the nucleic acids in the cells or tissues of the animal.

In some embodiments, a method of treating a patient with a disease is provided, comprising administering to the patient the polymer nanoparticle, wherein the polymer nanoparticle further comprises a drug payload, such as a polynucleotide or a protein payload, or a small molecule therapeutic or luminescent molecule payload, and treating the disease in the patient.

In various embodiments, any suitable route for administration of the polymer nanoparticles associated with nucleic acid constructs, or for the method of treatment can be used including parenteral administration or oral administration. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques. In other embodiments, oral or pulmonary routes of administration can be used.

In various embodiments, payloads may be combined with the polymer nanoparticles compositions using any or all of covalent bonds, electrostatic interactions, and ligand affinity interactions. In one aspect, covalent bonding methods include the use of EDC/NHS to form stable amide bonds between the payload and the polymer nanoparticles for improved stability (both “on the shelf” and in vivo), ease of separation and extraction, and sensitive detection. In another illustrative aspect, electrostatic bonding methods include the use of cationic polymer nanoparticles that electrostatically complex with the payload. In another embodiment, ligand affinity bonding includes the use of ligands such as avidin and biotin, both covalently bonded to the polymer nanoparticles and the payload via EDC/NHS chemistry to yield the stable combination of the payload and the polymer nanoparticles.

It will be appreciated that RAFT polymerization is generally known in the art. Suitable reagents, monomers, and conditions for RAFT polymerization previously investigated can be used in the copolymers, methods, and compositions described herein, such as those described in U.S. Pat. Nos. 9,006,193, 9,464,300, and 9,476,063, the disclosures of each of which are incorporated by reference in their entirety.

Chain transfer agents (CTAs) useful in connection with the present disclosure are known in the art. The identity of the CTA is not particularly limited. It will be appreciated that chain transfers steps that form the basis of RAFT polymerization involve a reversible transfer of a functional chain end-group (typically a thiocarbonylthio group, Z—C(═S)S—R) between chains and the propagating radicals. The overall process is comprised of the insertion of monomers between the R- and Z—C(═S)S-groups of a RAFT agent (CTA), which form the α and ω end-group of the majority of the resulting polymeric chains. Suitable CTAs for use in connection with the present disclosure include but are not limited to trithiocarbonates (Z═S-alkyl), dithiobenzoates (Z═Ph), dithiocarbamate (Z═N-alkyl), xanthates (Z═O-alkyl), and the like. (See, Sébastien Perrier, Macromolecules 2017 50 (19), 7433-7447). In some embodiments, RAFT copolymerization may be achieved using chain transfer agents (CTAs) containing one or more terminal carboxyl groups in order to obtain carboxy terminated polymers with ends available for bonding to the payload via the methods described above. In this embodiment, when the resulting mono or di-carboxy terminated polymer is dispersed in a low pH (e.g., a pH of less than 6) buffer, both ends of the polymer are exposed and available for labeling via EDC/NHS chemistry. In this embodiment, when the polymer is transferred to a physiological pH (˜pH 7), the core blocks self-assemble, encapsulating the payload in the hydrophobic core, to be released and exposed upon acidification in the endosomal compartment of a cell. In some embodiments, the first or second chain transfer agent can be selected from the group consisting of bis(carboxymethyl)trithiocarbonate, bis(2-amino-2-oxoethyl)trithiocarbonate, bis[4-(2-hydroxyethoxycarbonyl)benzyl]trithiocarbonate, 4-cyano-4-(ethylsulfanylthiocarbonyl) sulfanylvpentanoicacid, 4-cyano-4-((phenylcarbonothioyl)thio)pentanoic acid, and 4-cyano-4-[(dodecylsulfanylthiocarbonyl)sulfanyl]pentanoic acid, 4-cyano-4-(thiobenzoylthio)pentanoic acid, 2-cyano-2-propyl benzodithioate, cyanomethyl methyl(phenyl)carbamodithioate, 2-cyano-2-propyl dodecyl trithiocarbonate, 2-(dodecylthiocarbonothioylthio)-2-methylpropionic acid, cyanomethyl dodecyl trithiocarbonate, 2-cyano-2-propyl 4-cyanobenzodithioate, and the like.

In some embodiments, the block copolymer can be associated with a DNA molecule, in particular a nucleic acid construct of the present disclosure, via several methods including, electrostatic interaction, high affinity, non-covalent bond, avidin-streptavidin conjugation, or by direct covalent attachment through, for example, an amide bond. In some embodiments, the RAFT copolymer can be associated with a DNA molecule, in particular a nucleic acid construct of the present disclosure, via electrostatic interaction complexed with a biological molecule. In some embodiments, the block copolymer can be associated with a DNA molecule, in particular a nucleic acid construct of the present disclosure, via electrostatic interaction complexed with a biological molecule. In some embodiments, the block copolymer can be associated with a DNA molecule, in particular a nucleic acid construct of the present disclosure, via a high affinity, non-covalent bond, avidin-streptavidin conjugation. In some embodiments, the block copolymer can be associated with a DNA molecule, in particular a nucleic acid construct of the present disclosure, by direct covalent attachment through, for example, an amide bond.

The polymer nanoparticles described herein can be associated with a nucleic acid construct of the present disclosure via electrostatic interaction, avidin-streptavidin conjugation, or by direct covalent attachment. Exemplary interactions include: polymer nanoparticle (PNP) with positively charged corona in the case of electrostatic loading; nucleic acid constructs with negative charges due to the phosphate groups; electrostatically loaded PNP-nucleic acid construct complexes; carboxylate group on the terminal end of the polymer chains in the corona of the PNP; primary amine group on the 5′ end of the amine terminated nucleic acid construct; phosphate group on the 3′ end of the nucleic acid construct; amide bond formed in the direct amidification reaction between the amine terminal nucleic acid construct and the carboxylate terminated PNP; primary amine on the biotin bonding protein such as avidin; amide bond formed between the carboxylate group on the terminal end of the polymer chains in the corona of the PNP and the primary amine on the biotin bonding protein such as avidin; nucleic acid construct with a biotin functional group on the 5′ terminus; electrostatic coupling reaction that occurs when positively charged PNPs are mixed with negatively charged nucleic acid constructs; direct amidification reaction that is carried out via an EDA-NHC reaction between the carboxylate group on the terminal end of the polymer chains in the corona of the PNP and the primary amine on the amine terminated nucleic acid constructs; direct amidification reaction that is carried out via an EDA-NHC reaction between the carboxylate group on the terminal end of the polymer chains in the corona of the PNP and the primary amine on the biotin bonding protein such as avidin; coupling of the biotin on the 5′ end of the nucleic acid construct and the avidin conjugated to the carboxylate terminus on the corona of the PNPs.

It will be appreciated that tuning the parameters and properties of the block copolymers described herein can be advantageous to their use in the compositions and methods as described herein. Accordingly, the methods for preparing block copolymers either in singleton or in library format as described herein are capable of providing particular parameters and properties of the block copolymers.

In some embodiments, a single chain transfer agent can be used in the RAFT polymerization process in connection with the present disclosure. In some embodiments, for a block polymer having more than one block, one or more single chain transfer agents can be used in the RAFT polymerization process in connection with the present disclosure. In some embodiments, for a block polymer having two blocks, a first chain transfer agent and a second chain transfer agent (which can be the same or different) can be used at each step of the RAFT polymerization process in connection with the present disclosure. In some embodiments, for a block polymer having three blocks, a first chain transfer agent, a second chain transfer agent, and a third chain transfer agent (which can be the same or different) can be used at each step of the RAFT polymerization process in connection with the present disclosure.

It will be appreciated that a variety of solvents can be used in the RAFT polymerization method steps and purification steps described herein. Suitable solvents include, but are not limited to, 2-Chloroethanol, Acetic Acid (Glacial), Acetone, Acetonitrile, Acetophenone, Aniline, Benzaldehyde, Benzyl Acetate, Carbon disulfide, Cyclohexane, Cyclohexanol, Di(ethylene glycol), Di(propylene glycol), Diacetone alcohol, Diethyl ether, Dimethylsulfoxide, Ethanol, Ethyl acetate, Ethylene glycol, Formaldehyde (37% solution), Formamide, Formic acid, Formic acid (96%), HexaneIsobutanol, Isopropanol, Isopropyl acetate, Isopropyl ether, m-Cresol, Methanol, Methyl acetate, Methyl ethyl ketone, Mineral Oil, N,N-Dimethylformamide, n-Butanol, n-Octane, n-Propanol, Propylene glycol, Pyridine, t-Butanol, Tetrahydrofuran, Trifluoroacetic acid, water, and the like, and combinations thereof.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

### EXAMPLES

### Example 1

### Procedure for Block 1 Polymer Synthesis:

Diblock copolymers were synthesized using reversible addition-fragmentation chain transfer (RAFT) polymerization. To round bottom flasks were added the appropriate masses of monomer, chain transfer agent, thermal radical initiator, and solvent such that the mole ratio of chain transfer agent to initiator was 5:1 and the mole ratio of monomer to chain transfer agent was dependent on the target molecular weight. Example reagent masses for a block 1 reaction are listed in Table 2. After reagent addition the flasks were sparged with argon for 30 minutes, heated to 55° C. for 15 hours, then cooled in an ice bath and exposed to atmospheric oxygen. The synthesis product was purified by dialysis against DI water for 3 days with water changes about twice per day, followed by lyophilization for at least 3 days to remove water. The block 1 product was used as the macro chain transfer agent for the block 2 reaction, where it was chain extended with the desired monomer(s), solvent, and thermal radical initiator in polypropylene tubes such that the mole ratio of macro chain transfer agent to initiator was 1:1 and the mole ratio of monomer to macro chain transfer agent was dependent on the target molecular weight. Example amounts for block 2 synthesis are found in Table 2. After reagent addition the tubes were added to a vacuum oven, which was purged with argon for 45 minutes. Mixtures were reacted at about 60-65° C. for 24 hours, and cooled in an ice bath and exposed to atmospheric oxygen. The reaction product was purified by filtration or precipitation with appropriate solvents based on chemical composition.

Finally, the polymers were dried under vacuum or lyophilization and stored at room temperature for experimental use.

TABLE 2
Reagents and amounts used to synthesize an example polymer

| | | |
|---|---|---|
| Reagent | Purpose | Amount |

Block 1

| 2-aminoethylmethacrylamide (AEMA) | Monomer | 4445.75 | mg |
|---|---|---|---|
| (4-cyano-4-(((ethylthio)carbonothioyl)thio)pentanoicacid) (ECT) | Chain transfer | 63.26 | mg |
| | agent | | |
| 4,4′-Azobis(4-cyanovaleric acid) (ACVA) | Initiator | 16.12 | mg |
| Acetic buffer, pH 4-5 | Solvent | 27 | mL |

Block 2

| | | | |
|---|---|---|---|
| Butyl methacrylate (BMA) | Monomer | 24 | μL |
| Stock solution in acetic buffer of the block 1 reaction product at a | Macro chain | 83 | μL |

| concentration of 160 mg/mL. The ECT end groups (R & Z) were | | transfer agent |
|---|---|---|
| present on the ends of the p(AEMA) polymer synthesized in the | | |
| block 1 reaction, allowing it to function as a chain transfer agent. | | |
| 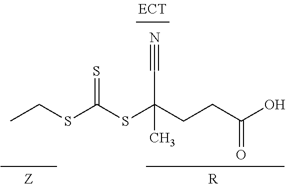 | For reference, here is ECT showing the R & Z groups on either side of the trithiocarbonyl group. | |

| Stock solution in acetic buffer of 4,4′-Azobis(4-cyanovaleric | Initiator | 32 | μL |
|---|---|---|---|
| acid) (ACVA) at a concentration of 12 mg/mL. | | | |
| Acetic buffer, pH 4-5 | Solvent | 160 | μL |

TABLE 3
Polymer composition

| | | | | | | | Reaction Output | |
|---|---|---|---|---|---|---|---|---|
| | Block 1 Composition | | | | | | Theoretical | Theorical |
| | | mCTA | Block 2 Composition | | | | Degree of | Overall Mw |
| ID | Monomer | Mw (kDa) | Monomer 1 | % Monomer 1 | Monomer 2 | % Monomer 2 | Polymerization | (kDa) |
| 1 | AEMA | 7.58 | BMA | 100 | HEMA | 0 | 15 | 9.715 |
| 2 | AEMA | 7.58 | BMA | 80 | HEMA | 20 | 15 | 9.679 |
| 3 | AEMA | 7.58 | BMA | 60 | HEMA | 40 | 15 | 9.642 |
| 4 | AEMA | 7.58 | BMA | 40 | HEMA | 60 | 15 | 9.606 |
| 5 | AEMA | 7.58 | BMA | 20 | HEMA | 80 | 15 | 9.57 |
| 6 | AEMA | 7.58 | BMA | 0 | HEMA | 100 | 15 | 9.534 |
| 7 | AEMA | 7.58 | BMA | 100 | HEMA | 0 | 20 | 10.426 |
| 8 | AEMA | 7.58 | BMA | 80 | HEMA | 20 | 20 | 10.378 |
| 9 | AEMA | 7.58 | BMA | 60 | HEMA | 40 | 20 | 10.329 |
| 10 | AEMA | 7.58 | BMA | 40 | HEMA | 60 | 20 | 10.281 |
| 11 | AEMA | 7.58 | BMA | 20 | HEMA | 80 | 20 | 10.233 |
| 12 | AEMA | 7.58 | BMA | 0 | HEMA | 100 | 20 | 10.185 |
| 13 | AEMA | 7.58 | BMA | 100 | HEMA | 0 | 30 | 11.848 |
| 14 | AEMA | 7.58 | BMA | 80 | HEMA | 20 | 30 | 11.775 |
| 15 | AEMA | 7.58 | BMA | 60 | HEMA | 40 | 30 | 11.703 |
| 16 | AEMA | 7.58 | BMA | 40 | HEMA | 60 | 30 | 11.631 |
| 17 | AEMA | 7.58 | BMA | 20 | HEMA | 80 | 30 | 11.558 |
| 18 | AEMA | 7.58 | BMA | 0 | HEMA | 100 | 30 | 11.486 |
| 19 | AEMA | 7.58 | BMA | 100 | HEMA | 0 | 60 | 16.114 |
| 20 | AEMA | 7.58 | BMA | 80 | HEMA | 20 | 60 | 15.969 |
| 21 | AEMA | 7.58 | BMA | 60 | HEMA | 40 | 60 | 15.824 |
| 22 | AEMA | 7.58 | BMA | 40 | HEMA | 60 | 60 | 15.68 |
| 23 | AEMA | 7.58 | BMA | 20 | HEMA | 80 | 60 | 15.535 |
| 24 | AEMA | 7.58 | BMA | 0 | HEMA | 100 | 60 | 15.39 |
| 25 | AEMA | 14.38 | BMA | 100 | HEMA | 0 | 30 | 18.642 |
| 26 | AEMA | 14.38 | BMA | 80 | HEMA | 20 | 30 | 18.57 |
| 27 | AEMA | 14.38 | BMA | 60 | HEMA | 40 | 30 | 18.497 |
| 28 | AEMA | 14.38 | BMA | 40 | HEMA | 60 | 30 | 18.425 |
| 29 | AEMA | 14.38 | BMA | 20 | HEMA | 80 | 30 | 18.353 |
| 30 | AEMA | 14.38 | BMA | 0 | HEMA | 100 | 30 | 18.28 |
| 31 | AEMA | 14.38 | BMA | 100 | HEMA | 0 | 40 | 20.064 |
| 32 | AEMA | 14.38 | BMA | 80 | HEMA | 20 | 40 | 19.968 |
| 33 | AEMA | 14.38 | BMA | 60 | HEMA | 40 | 40 | 19.871 |
| 34 | AEMA | 14.38 | BMA | 40 | HEMA | 60 | 40 | 19.775 |
| 35 | AEMA | 14.38 | BMA | 20 | HEMA | 80 | 40 | 19.678 |
| 36 | AEMA | 14.38 | BMA | 0 | HEMA | 100 | 40 | 19.582 |
| 37 | AEMA | 14.38 | BMA | 100 | HEMA | 0 | 60 | 22.908 |
| 38 | AEMA | 14.38 | BMA | 80 | HEMA | 20 | 60 | 22.763 |
| 39 | AEMA | 14.38 | BMA | 60 | HEMA | 40 | 60 | 22.619 |
| 40 | AEMA | 14.38 | BMA | 40 | HEMA | 60 | 60 | 22.474 |
| 41 | AEMA | 14.38 | BMA | 20 | HEMA | 80 | 60 | 22.329 |
| 42 | AEMA | 14.38 | BMA | 0 | HEMA | 100 | 60 | 22.185 |
| 43 | AEMA | 14.38 | BMA | 100 | HEMA | 0 | 120 | 31.44 |
| 44 | AEMA | 14.38 | BMA | 80 | HEMA | 20 | 120 | 31.151 |
| 45 | AEMA | 14.38 | BMA | 60 | HEMA | 40 | 120 | 30.861 |
| 46 | AEMA | 14.38 | BMA | 40 | HEMA | 60 | 120 | 30.572 |
| 47 | AEMA | 14.38 | BMA | 20 | HEMA | 80 | 120 | 30.282 |
| 48 | AEMA | 14.38 | BMA | 0 | HEMA | 100 | 120 | 29.993 |
| 49 | AEMA | 19.65 | BMA | 100 | HEMA | 0 | 45 | 26.047 |
| 50 | AEMA | 19.65 | BMA | 80 | HEMA | 20 | 45 | 25.938 |
| 51 | AEMA | 19.65 | BMA | 60 | HEMA | 40 | 45 | 25.83 |
| 52 | AEMA | 19.65 | BMA | 40 | HEMA | 60 | 45 | 25.721 |
| 53 | AEMA | 19.65 | BMA | 20 | HEMA | 80 | 45 | 25.613 |
| 54 | AEMA | 19.65 | BMA | 0 | HEMA | 100 | 45 | 25.504 |
| 55 | AEMA | 19.65 | BMA | 100 | HEMA | 0 | 60 | 28.18 |
| 56 | AEMA | 19.65 | BMA | 80 | HEMA | 20 | 60 | 28.035 |
| 57 | AEMA | 19.65 | BMA | 60 | HEMA | 40 | 60 | 27.89 |
| 58 | AEMA | 19.65 | BMA | 40 | HEMA | 60 | 60 | 27.746 |
| 59 | AEMA | 19.65 | BMA | 20 | HEMA | 80 | 60 | 27.601 |
| 60 | AEMA | 19.65 | BMA | 0 | HEMA | 100 | 60 | 27.456 |
| 61 | AEMA | 19.65 | BMA | 100 | HEMA | 0 | 90 | 32.446 |
| 62 | AEMA | 19.65 | BMA | 80 | HEMA | 20 | 90 | 32.229 |
| 63 | AEMA | 19.65 | BMA | 60 | HEMA | 40 | 90 | 32.012 |
| 64 | AEMA | 19.65 | BMA | 40 | HEMA | 60 | 90 | 31.795 |
| 65 | AEMA | 19.65 | BMA | 20 | HEMA | 80 | 90 | 31.578 |
| 66 | AEMA | 19.65 | BMA | 0 | HEMA | 100 | 90 | 31.361 |
| 67 | AEMA | 19.65 | BMA | 100 | HEMA | 0 | 180 | 45.244 |
| 68 | AEMA | 19.65 | BMA | 80 | HEMA | 20 | 180 | 44.81 |
| 69 | AEMA | 19.65 | BMA | 60 | HEMA | 40 | 180 | 44.376 |
| 70 | AEMA | 19.65 | BMA | 40 | HEMA | 60 | 180 | 43.941 |
| 71 | AEMA | 19.65 | BMA | 20 | HEMA | 80 | 180 | 43.507 |
| 72 | AEMA | 19.65 | BMA | 0 | HEMA | 100 | 180 | 43.073 |
| 73 | AEMA | 23.96 | BMA | 100 | HEMA | 0 | 60 | 32.494 |
| 74 | AEMA | 23.96 | BMA | 80 | HEMA | 20 | 60 | 32.349 |
| 75 | AEMA | 23.96 | BMA | 60 | HEMA | 40 | 60 | 32.204 |
| 76 | AEMA | 23.96 | BMA | 40 | HEMA | 60 | 60 | 32.06 |
| 77 | AEMA | 23.96 | BMA | 20 | HEMA | 80 | 60 | 31.915 |
| 78 | AEMA | 23.96 | BMA | 0 | HEMA | 100 | 60 | 31.77 |
| 79 | AEMA | 23.96 | BMA | 100 | HEMA | 0 | 80 | 35.338 |
| 80 | AEMA | 23.96 | BMA | 80 | HEMA | 20 | 80 | 35.145 |
| 81 | AEMA | 23.96 | BMA | 60 | HEMA | 40 | 80 | 34.952 |
| 82 | AEMA | 23.96 | BMA | 40 | HEMA | 60 | 80 | 34.759 |
| 83 | AEMA | 23.96 | BMA | 20 | HEMA | 80 | 80 | 34.566 |
| 84 | AEMA | 23.96 | BMA | 0 | HEMA | 100 | 80 | 34.373 |
| 85 | AEMA | 23.96 | BMA | 100 | HEMA | 0 | 120 | 41.026 |
| 86 | AEMA | 23.96 | BMA | 80 | HEMA | 20 | 120 | 40.736 |
| 87 | AEMA | 23.96 | BMA | 60 | HEMA | 40 | 120 | 40.447 |
| 88 | AEMA | 23.96 | BMA | 40 | HEMA | 60 | 120 | 40.157 |
| 89 | AEMA | 23.96 | BMA | 20 | HEMA | 80 | 120 | 39.868 |
| 90 | AEMA | 23.96 | BMA | 0 | HEMA | 100 | 120 | 39.578 |
| 91 | AEMA | 23.96 | BMA | 100 | HEMA | 0 | 240 | 58.09 |
| 92 | AEMA | 23.96 | BMA | 80 | HEMA | 20 | 240 | 57.511 |
| 93 | AEMA | 23.96 | BMA | 60 | HEMA | 40 | 240 | 56.932 |
| 94 | AEMA | 23.96 | BMA | 40 | HEMA | 60 | 240 | 56.353 |
| 95 | AEMA | 23.96 | BMA | 20 | HEMA | 80 | 240 | 55.774 |
| 96 | AEMA | 23.96 | BMA | 0 | HEMA | 100 | 240 | 55.195 |
| 97 | AEMA | 35.316 | BMA | 100 | HEMA | 0 | 70.6 | 45.354 |
| 98 | AEMA | 21.231 | BMA | 0 | HEMA | 100 | 35.3 | 25.829 |
| 99 | AEMA | 21.428 | BMA | 33 | HEMA | 67 | 18.9 | 23.959 |
| 100 | AEMA | 21.231 | BMA | 33 | HEMA | 67 | 38.6 | 26.408 |
| 101 | AEMA | 21.231 | BMA | 33 | HEMA | 67 | 113 | 36.347 |
| 102 | AEMA | 27.826 | BMA | 67 | HEMA | 33 | 57.8 | 35.815 |
| 103 | AEMA | 35.316 | BMA | 67 | HEMA | 33 | 68.8 | 44.822 |
| 104 | AEMA | 21.428 | BMA | 100 | HEMA | 0 | 20.4 | 24.323 |
| 105 | AEMA | 21.428 | BMA | 0 | HEMA | 100 | 19.1 | 23.914 |
| 106 | AEMA | 21.428 | BMA | 33 | HEMA | 67 | 57.7 | 29.166 |
| 107 | AEMA | 21.231 | BMA | 100 | HEMA | 0 | 38.1 | 26.649 |

### Procedure for Particle Size Measurement Using Dynamic Light Scattering:

Di-block polymers in a dry solid state were rehydrated using ultrapure water or suitable aqueous buffer to a concentration in the range of about 1-40 mg/mL, utilizing a combination of agitation by orbital shaker and sonication to disperse the polymer into nanoparticles. Solutions were centrifuged for 5 minutes to settle any dust or large aggregates present. Aliquots were transferred to a flat-bottom well plate suitable for measurement and diluted tenfold, resulting in a polymer concentration in the range of about 0.1-4 mg/mL. The plate was measured using a Wyatt DynaPro Plate Reader III utilizing 7 acquisitions per well. Suitable data filters were applied to the resulting autocorrelation functions to remove low-quality acquisitions. Representative data filter parameters include a baseline acceptance criterion of 1±0.05, a minimum amplitude of 0.05, and a maximum sum-of-squares error of 100. The Z-average diameter was used to provide the hydrodynamic diameter.

TABLE 4
Physical Characterization of PNPs

| | Physical Characterization | | |
|---|---|---|---|
| | | | Particle Diameter, |
| | Particle Diameter, | Zeta | Loaded with |
| | Unloaded | Potential | 19.5 kb plasmid |
| ID | (nm) | (mV) | (nm) |
| 1 | 250.0 | 58.1 | 532.0 |
| 2 | 239.9 | 51.4 | 891.3 |
| 3 | 18.3 | 55.3 | 1575.3 |
| 4 | 5.4 | 48.6 | 1128.3 |
| 5 | 184.0 | 48.3 | 409.5 |
| 6 | 5.6 | 24.2 | 912.3 |
| 7 | 528.1 | 57.6 | 1042.2 |
| 8 | | 53.4 | 1451.0 |
| 9 | 8.6 | 55 | 476.8 |
| 10 | 236.5 | 55.7 | 804.3 |
| 11 | 29.2 | 43.5 | 1556.0 |
| 12 | 5.4 | 21.1 | 1725.9 |
| 13 | 442.1 | 55.5 | 516.0 |
| 14 | 91.2 | 54.3 | 849.2 |
| 15 | 127.3 | 54.3 | 1930.0 |
| 16 | 171.0 | 56.3 | 1761.4 |
| 17 | 273.0 | 54.1 | 364.9 |
| 18 | | 22.2 | 1175.3 |
| 19 | 251.6 | 54.8 | 1258.4 |
| 20 | 267.6 | 52.5 | 2041.1 |
| 21 | 250.5 | 54.8 | 723.5 |
| 22 | N/A | 45 | N/A |
| 23 | 79.3 | 41 | 916.7 |
| 24 | N/A | 20.7 | N/A |
| 25 | 556.9 | 54.1 | 496.8 |
| 26 | 45.5 | 52.6 | 362.6 |
| 27 | 233.1 | 50.8 | 289.0 |
| 28 | 160.4 | 47.2 | 323.4 |
| 29 | 53.3 | 43.9 | 385.9 |
| 30 | 8.6 | 28.9 | 287.4 |
| 31 | 238.7 | 51.5 | 306.0 |
| 32 | 230.0 | 49.7 | 365.4 |
| 33 | 194.3 | 47.5 | 301.7 |
| 34 | 8.0 | 50.6 | 344.3 |
| 35 | 11.1 | 36.1 | 289.1 |
| 36 | N/A | 43.2 | N/A |
| 37 | 537.2 | 56.9 | 320.2 |
| 38 | 268.0 | 54.4 | 377.0 |
| 39 | 39.4 | 26.3 | 369.0 |
| 40 | 125.7 | 32.4 | 431.8 |
| 41 | N/A | 28.1 | N/A |
| 42 | N/A | 23.4 | N/A |
| 43 | 213.4 | 53.5 | 304.0 |
| 44 | 69.8 | 44.6 | 400.5 |
| 45 | 175.0 | 50 | 331.1 |
| 46 | 0.6 | 50.8 | 282.5 |
| 47 | N/A | 39.6 | N/A |
| 48 | N/A | 37.8 | N/A |
| 49 | 277.5 | 45.5 | 557.8 |
| 50 | 55.2 | 44.9 | 456.7 |
| 51 | 155.0 | 45 | 461.5 |
| 52 | 10.0 | 29 | 481.4 |
| 53 | 7.7 | 29.3 | 435.4 |
| 54 | 7.9 | 40.2 | 444.3 |
| 55 | 237.1 | 50.2 | 361.8 |
| 56 | 145.5 | 48.2 | 317.9 |
| 57 | 8.4 | 42.5 | 379.5 |
| 58 | 12.5 | 36 | 394.1 |
| 59 | 8.3 | 41.3 | 354.7 |
| 60 | 9.1 | 37.9 | 309.5 |
| 61 | 231.2 | 47.4 | 323.0 |
| 62 | 140.4 | 41.8 | 459.1 |
| 63 | 86.9 | 40.8 | 435.2 |
| 64 | 23.7 | 37 | 401.3 |
| 65 | 17.3 | 33.4 | 364.1 |
| 66 | N/A | 41.6 | N/A |
| 67 | 181.6 | 48 | 315.8 |
| 68 | N/A | 43.5 | 499.8 |
| 69 | N/A | 42.3 | N/A |
| 70 | 60.6 | 47 | 656.6 |
| 71 | N/A | 47 | N/A |
| 72 | N/A | 39.3 | N/A |
| 73 | 257.7 | 49.9 | 360.8 |
| 74 | 75.3 | 49.9 | 337.8 |
| 75 | 16.5 | 46.5 | 303.6 |
| 76 | 9.4 | 50.7 | 483.2 |
| 77 | 8.6 | 36.8 | 297.7 |
| 78 | N/A | 29.7 | N/A |
| 79 | 210.8 | 48.1 | 315.2 |
| 80 | 63.3 | 46.2 | 269.0 |
| 81 | 14.2 | 44.5 | 287.1 |
| 82 | 9.3 | 32.4 | 324.5 |
| 83 | 9.1 | 39.9 | 315.1 |
| 84 | 9.4 | 32.4 | 364.0 |
| 85 | 230.0 | 48.9 | 330.1 |
| 86 | 126.3 | 50 | 228.9 |
| 87 | 11.6 | 44.2 | 265.5 |
| 88 | 11.7 | 43.9 | 245.0 |
| 89 | 12.4 | 47 | 312.1 |
| 90 | 11.5 | 39.1 | 376.1 |
| 91 | 193.8 | 49.3 | 305.8 |
| 92 | N/A | 39.5 | N/A |
| 93 | 12.5 | 45 | 328.7 |
| 94 | 18.2 | 44.6 | 321.0 |
| 95 | N/A | 41.8 | N/A |
| 96 | N/A | 44.8 | N/A |
| 97 | 93.7 | N/A | 301.5 |
| 98 | 298.6 | N/A | 454.8 |
| 99 | 248.4 | N/A | 306 |
| 100 | 288.2 | N/A | 311.1 |
| 101 | 378.1 | N/A | 395.8 |
| 102 | 206.6 | N/A | 217.3 |
| 103 | 226.4 | N/A | 258.1 |
| 104 | 85.9 | N/A | 454.5 |
| 105 | N/A | N/A | N/A |
| 106 | 314.5 | N/A | 401.1 |
| 107 | 44.8 | N/A | N/A |

### Example 2

### Ribogreen Assay for Calculating Encapsulation Efficiency

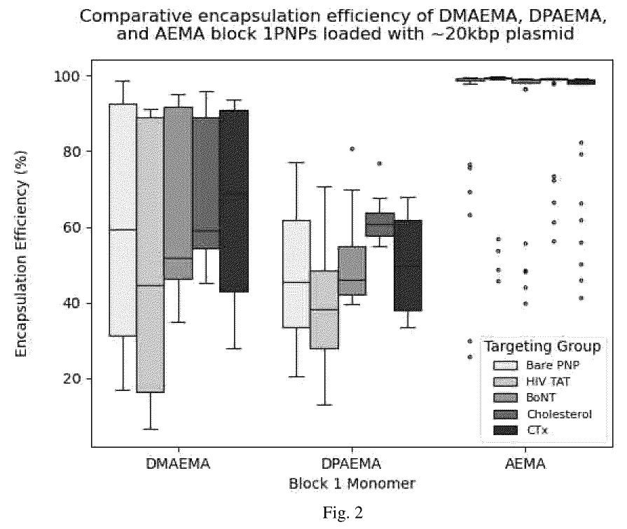

Polymers loaded at 3 different PNP:pDNA weight ratios 15:1, 25:1 and 10:1 with 150 ng of Plasmid DNA in a 96 well plate. After 30 minutes of incubation 100 μL of Ribogreen™ Reagent (1:1000 dilution) is added to the samples for a total volume of 200 μL. The samples are scanned at ex. 480 em. 520 for fluorescence of the DNA intercalating dye. The fluorescence intensity is correlated to a standard curve of the DNA to calculate the encapsulation efficiency. DNA that has been shielded by the PNP will not fluoresce leading to lower fluorescence intensity for PNPs with greater encapsulation efficiency. The results of the 15:1 ratio are shown in Table 5. Results are also shown in FIG. 2.

### Procedure for In-Vitro Transfection Efficiency and Cell Viability Testing:

Polymer nanoparticles (PNPs) were tested in-vitro on hTERT Schwann ipn97.4 cells. Cells were cultured in DMEM with an added 10% of FBS and 1% of Pen-Strep and kept below a passage number of 10. These cells were plated in Poly-Lysine coated 96 well plates at 25,000 cells per well. Then, set to incubate (37° C., 5% CO₂) for 24 hours to allow cells to become confluent. On treatment day, the plasmid DNA was added to the PNPs at different weight ratios (i.e., 25:1, 40:1, 50:1) and incubated for at least 4 hours at RT to ensure encapsulation. See table below for example calculation for PNP prep. 10 μL of this PNP encapsulated cDNA solution was added to the appropriate wells of the Schwann cell plate and placed in the incubator (37° C., 5% CO₂) to allow time for transfection to occur. At 48 hours post treatment, the plate was imaged on the Cytation 10 to count number to total cells per well along with number of cDNA transfected cells (via GFP reporter). With these values the transfection efficiency and viability was calculated for each PNP. For another analysis method this plate was also ran with flow cytometry.

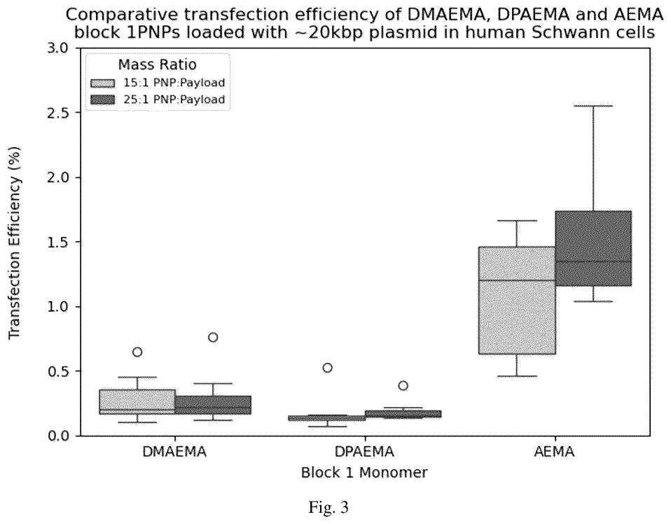

After imaging was complete, the cells were immediately stained with a Zombie Violet viability dye and fixed with 4% PFA. The viability dye was added at a 1:100 dilution from the stock and incubated with the cells at RT for 20 minutes as per the vendor's recommendations. After staining was done, the cells were washed with PBS and fixed at a 1:1 ratio with 4% PFA. This was incubated at RT for 10 minutes and the plate was centrifuged to remove remaining fixative. The ending sample resuspension was 80 uL to accommodate a 60 uL acquisition on the cytometer. On the flow cytometer both the GFP payload and Zombie Violet signal was tracked to calculate transfection efficiency percentages and cell viability. These values were used as a comparison to the image cytometer results to create a clearer picture of how well the PNPs can deliver the payload. The results for the 10:1 ratio are shown in Table 5. Results are also shown in FIG. 3

TABLE 5
Functional Characterization of PNPs 1-107

| | Functional Characterization of PNPs delivering | | |
|---|---|---|---|
| | a 19.5 kbp plasmid to human Schwann cells. | | |
| | Encapsulation | Transfection | Cell |
| | Efficiency | Efficiency | Viability |
| ID | (%) | (%) | (%) |
| 1 | 101.0 | 9.6 | 82.6 |
| 2 | 100.3 | 7.2 | 82.5 |
| 3 | 100.7 | 6.0 | 84.3 |
| 4 | 100.3 | 9.9 | 78.5 |
| 5 | 100.3 | 8.9 | 80.3 |
| 6 | 100.3 | 5.8 | 85.1 |
| 7 | 101.0 | 7.0 | 84.0 |
| 8 | 100.3 | 11.3 | 77.3 |
| 9 | 100.3 | 8.5 | 82.8 |
| 10 | 100.0 | 5.5 | 83.0 |
| 11 | 100.0 | 7.5 | 80.9 |
| 12 | 100.0 | 8.4 | 81.4 |
| 13 | 101.0 | 6.8 | 85.1 |
| 14 | 100.0 | 5.0 | 85.7 |
| 15 | 100.0 | 6.9 | 83.0 |
| 16 | 100.0 | 10.2 | 76.4 |
| 17 | 100.0 | 6.6 | 83.6 |
| 18 | 100.0 | 5.8 | 83.7 |
| 19 | 101.0 | 5.9 | 85.8 |
| 20 | 100.0 | 7.8 | 81.1 |
| 21 | 100.0 | 7.1 | 83.6 |
| 22 | N/A | N/A | N/A |
| 23 | 100.0 | 5.3 | 86.4 |
| 24 | N/A | N/A | N/A |
| 25 | 101.0 | 9.1 | 74.9 |
| 26 | 100.3 | 8.7 | 64.9 |
| 27 | 100.3 | 9.2 | 69.8 |
| 28 | 100.0 | 8.1 | 79.3 |
| 29 | 100.7 | 10.3 | 75.9 |
| 30 | 101.0 | 9.3 | 75.9 |
| 31 | 101.0 | 2.3 | 90.6 |
| 32 | 101.0 | 11.3 | 75.9 |
| 33 | 101.0 | 11.6 | 69.3 |
| 34 | 100.3 | 9.8 | 69.5 |
| 35 | 100.3 | 7.1 | 72.9 |
| 36 | N/A | N/A | N/A |
| 37 | 101.0 | 9.8 | 78.7 |
| 38 | 100.7 | 9.7 | 77.1 |
| 39 | 100.3 | 9.9 | 71.9 |
| 40 | 100.0 | 3.4 | 87.1 |
| 41 | N/A | N/A | N/A |
| 42 | N/A | N/A | N/A |
| 43 | 101.0 | 9.8 | 79.4 |
| 44 | 100.0 | N/A | N/A |
| 45 | 100.0 | 11.0 | 76.1 |
| 46 | 100.7 | 8.8 | 77.9 |
| 47 | N/A | N/A | N/A |
| 48 | N/A | N/A | N/A |
| 49 | 100.7 | 5.1 | 84.6 |
| 50 | 101.0 | 7.7 | 75.2 |
| 51 | 101.0 | 13.4 | 79.6 |
| 52 | 101.0 | 12.5 | 74.3 |
| 53 | 101.0 | 6.3 | 82.6 |
| 54 | 100.0 | 9.2 | 76.0 |
| 55 | 101.0 | 10.5 | 79.1 |
| 56 | 101.0 | 9.5 | 83.0 |
| 57 | 100.7 | 7.4 | 80.1 |
| 58 | 100.7 | 9.7 | 76.7 |
| 59 | 100.7 | 8.9 | 82.6 |
| 60 | 100.3 | 6.1 | 83.8 |
| 61 | 101.0 | 6.8 | 79.5 |
| 62 | 100.7 | 11.7 | 69.2 |
| 63 | 100.7 | 10.3 | 79.8 |
| 64 | 100.3 | 9.6 | 79.8 |
| 65 | 100.3 | 6.3 | 79.1 |
| 66 | N/A | N/A | N/A |
| 67 | 101.0 | 10.6 | 67.0 |
| 68 | 100.7 | 8.5 | 79.5 |
| 69 | N/A | N/A | N/A |
| 70 | 100.0 | 9.9 | 70.7 |
| 71 | N/A | N/A | N/A |
| 72 | N/A | N/A | N/A |
| 73 | 101.0 | 6.1 | 77.3 |
| 74 | 101.0 | 10.2 | 76.9 |
| 75 | 101.0 | 10.5 | 79.0 |
| 76 | 101.0 | N/A | N/A |
| 77 | 101.0 | 4.5 | 89.2 |
| 78 | N/A | N/A | N/A |
| 79 | 101.0 | 10.2 | 70.6 |
| 80 | 101.0 | 12.7 | 80.3 |
| 81 | 101.0 | N/A | N/A |
| 82 | 101.0 | 8.2 | 76.6 |
| 83 | 101.0 | 12.0 | 69.2 |
| 84 | 100.7 | 13.7 | 81.7 |
| 85 | 101.0 | | |
| 86 | 101.0 | 7.5 | 76.3 |
| 87 | 101.0 | 7.8 | 82.5 |
| 88 | 101.0 | 8.5 | 85.6 |
| 89 | 101.0 | 11.0 | 82.6 |
| 90 | 100.7 | 9.2 | 75.7 |
| 91 | 101.0 | 12.1 | 73.9 |
| 92 | N/A | N/A | N/A |
| 93 | 100.7 | 10.9 | 76.4 |
| 94 | 101.0 | 11.6 | 76.7 |
| 95 | N/A | N/A | N/A |
| 96 | N/A | N/A | N/A |
| 97 | 101.7 | 3.57 | 93.14 |
| 98 | 101.33 | 2.45 | 94.99 |
| 99 | 101.00 | 3.40 | 94.43 |
| 100 | 101.33 | 3.35 | 96.18 |
| 101 | 101.33 | 2.31 | 94.35 |
| 102 | 101.33 | 2.76 | 95.44 |
| 103 | 101.33 | 3.72 | 94.31 |
| 104 | 101.67 | 2.73 | 88.65 |
| 105 | N/A | N/A | N/A |
| 106 | 101.33 | 2.71 | 86.63 |
| 107 | 92.395 | N/A | 87.68 |

TABLE 6
PNP Treatment Prep Calculation

| | | | Volume per |
|---|---|---|---|
| | | | well (uL) |
| PNP stock Concentration (mg/mL) | 2 | | |
| pDNA stock concentration (ug/uL) | 0.2548 | | |
| PNP:pDNA Weight Ratio | 25 | PBS | 7.54 |
| PNP Final Concentration (mg/mL) | 0.0375 | PNP | 1.88 |
| # of Replicates | 3 | pDNA | 0.59 |
| PNP:PEG Cholesterol Weight | 30:1 | | |
| Ratio | | | |

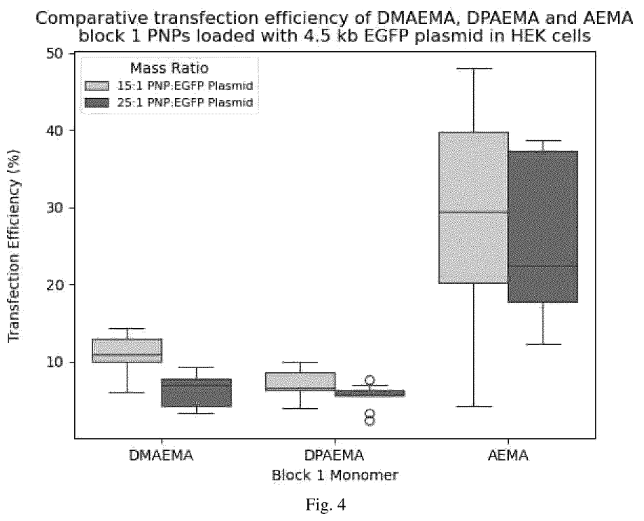

* For the HEK cell data, (see FIG. 4), the PNPs were loaded in Opti-MEM instead of PBS and incubated for 30 min instead of 4.

### Example 3

PNP 104 was prepared and used in this example. PNP 104 had characteristics according to Table 7.

TABLE 7

| PNP ID | 104 | 104 |
|---|---|---|
| Composition | p(AEMA)-b-p(BMA) | p(AEMA)-b-p(BMA) |
| Approximate | 3:1 | 3:1 |
| Block1:Block 2 | | |
| mole ratio | | |
| Block 1 target DP | 57.91 | 57.91 |
| Block 2 target DP | 20.36 | 20.83 |

DP = degree of polymerization

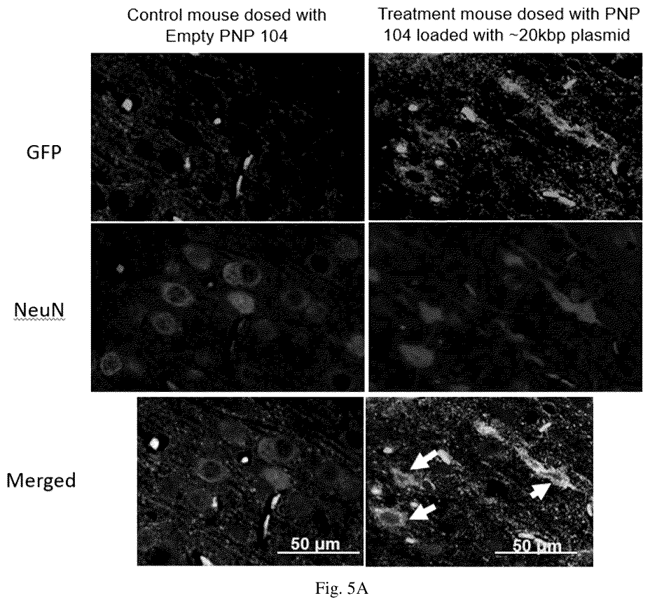

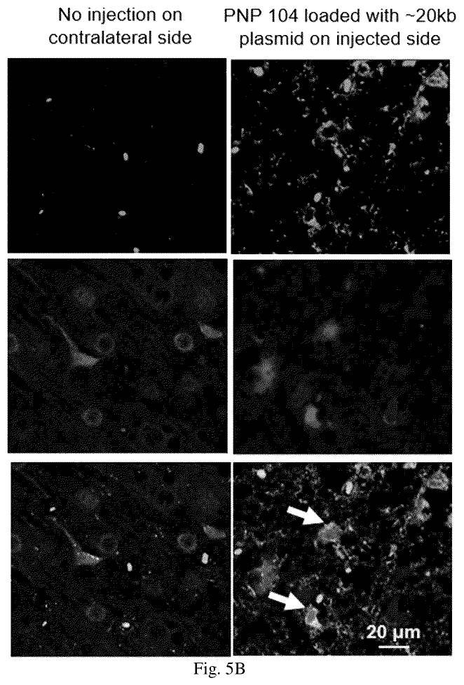

The PNPs were administered to 12 Sprague Dawley rats to assess expression and cell specific expression. Both the PNPs were loaded with a GFP plasmid (19.5 kbp) and administered to wild-type Sprague Dawley rats via intracerebroventricular (ICV) injection at a 25:1 PNP:DNA ratio for a targeted dose of 0.5 mg/kg to investigate cell-specific expression of the delivered plasmid. Brain tissue was harvested after 72 hours of PNP treatment and immunofluorescence was performed. Multiple GFP positive neural cells (stained with NeuN) were seen in the brain near the site of injection, but no GFP positive astrocytes were detected (stained with GFAP). Results are shown in FIGS. 5A-B.

### Example 4

The ability of the PNPs to deliver a large 20.3 kbp eGFP expressing plasmid was tested. PNPs at three different concentrations, 0.0075 mg/ml, 0.015 mg/ml and 0.03 mg/ml, were loaded with 300 ng of the 20.3 kbp plasmid and administered to HEK293T cells in vitro. Lipofectamine was used as a positive control. Positive GFP expression was observed in the HEK293 cells at 48 hr and 72 hr post transfection.

1. A block copolymer comprising
a first block homopolymer of monomer units, wherein each monomer unit is represented by formula I: 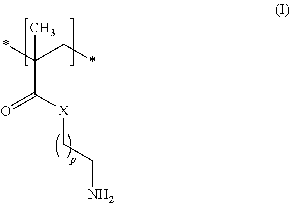
or a salt thereof, wherein:
X is —O— or —NH—; and
p is 0 or an integer selected from 1-3, and
wherein each * represents a point of covalent attachment to the rest of the block copolymer; and
a second block of either:
(i) a homopolymer of monomer units, wherein each monomer unit is represented by formula II: 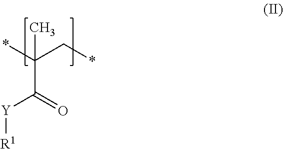
or a salt thereof, wherein:
Y is —O— or —NH—; and
R¹ is C₁-C₆ alkyl or glucose, wherein each hydrogen atom in C₁-C₆ alkyl is optionally substituted by hydroxy, and
wherein each * represents a point of covalent attachment to the rest of the block copolymer; or
(ii) a copolymer comprising two or more monomer units each individually represented by formula II: 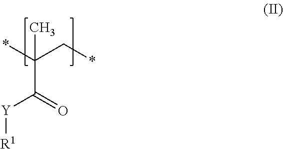
or a salt thereof, wherein:
Y is —O— or —NH—; and
R¹ is C₁-C₆ alkyl or glucose, wherein each hydrogen atom in C₁-C₆ alkyl is optionally substituted by hydroxy, and
wherein each * represents a point of covalent attachment to the rest of the block copolymer; and
wherein the first block is at least about 15 mol % of the block copolymer.

2. (canceled)

3. The block copolymer of claim 1, wherein p is 1.

4. (canceled)

5. The block copolymer of claim 1, wherein the second block comprises a homopolymer of a monomer unit represented by formula II.

6-8. (canceled)

9. The block copolymer of claim 1, wherein R¹ is C₂-C₆ alkyl.

10. (canceled)

11. The block copolymer of claim 5, wherein R¹ is 2-ethanol.

12. The block copolymer of claim 5, wherein R¹ is C₃-C₆ alkyl.

13. (canceled)

14. The block copolymer of claim 12, wherein R¹ is unsubstituted butyl.

15. The block copolymer of claim 1, wherein Y is —NH—.

16. The block copolymer of claim 15, wherein R¹ is glucose.

17. The block copolymer of claim 1, wherein the second block comprises a copolymer comprising a first monomer unit represented by formula II and a second monomer unit represented by formula II.

18. The block copolymer of claim 17, wherein the Y is —O— in the first monomer unit.

19. The block copolymer of claim 17, wherein the Y is —O— in the second monomer unit.

20. The block copolymer of claim 17, wherein R¹ is C₁-C₆ alkyl in the first monomer unit.

21. The block copolymer of claim 17, wherein R¹ is C₁-C₆ alkyl in the second monomer unit.

22. The block copolymer of claim 17, wherein R¹ is ethyl optionally substituted by hydroxy in the first monomer unit.

23. The block copolymer of claim 17, wherein R¹ is butyl in the first monomer unit.

24. The block copolymer of claim 17, wherein R¹ is butyl in the second monomer unit.

25. A block copolymer comprising a first block and a second block, wherein:
the first block comprise a homopolymer poly aminoethyl methacrylamide (AEMA), and
the second block comprises either:
(a) a homopolymer of polybutylmethacrylate (BMA), hydroxyethylmethacrylate (HEMA), or 2-deoxy-2-methacrylamido glucopyranose (MAG) or
(b) a copolymer of at least two of BMA, HEMA, and MAG, wherein the first block has a molecular weight (Mw) of at least 9,000 Da.

26. The block copolymer of claim 25, wherein the second block comprises a homopolymer of BMA.

27. The block copolymer of claim 25, wherein the second block comprises a homopolymer of HEMA.

28. The block copolymer of claim 25, wherein the second block comprises a copolymer of BMA and HEMA.

29. The block copolymer of claim 1, wherein the first block is at least about 20 mol % of the block copolymer.

30-44. (canceled)

45. A polymer nanoparticle comprising:
a block copolymer according to claim 1,
wherein the nanoparticle has a hydrodynamic diameter of about 30 nm to about 500 nm.

46-47. (canceled)

48. The polymer nanoparticle of claim 45, wherein the nanoparticle has a hydrodynamic diameter of about 50 nm to about 300 nm.

49-50. (canceled)

51. The polymer nanoparticle of claim 45, wherein the nanoparticle has a size poly dispersity index (PDI) of about 0.15 to about 0.3, as measured using DLS.

52-58. (canceled)

59. A composition comprising:
a polymer nanoparticle according to claim 45, and
a nucleic acid complexed to the polymer nanoparticle.

60-64. (canceled)

65. The composition of claim 59, wherein the nucleic acid is about 15 kbp to about 25 kbp.

66. (canceled)

67. The composition of claim 59, wherein the encapsulation efficiency is at least about 75%.

68. The composition of claim 59, wherein the transfection efficiency of the composition into cells is at least about 1%

69. The composition of claim 59, wherein the cell viability of the cells after transfection is at least about 70%.

70-71. (canceled)
